(19) United States
(12) Patent Application Publication
(10) Pub. No.: US 2018/0271997 A1
(43) Pub. Date: Sep. 27, 2018

(54) Methods and Reagents to Treat Tumor and Cancer

(71) Applicant: Tianxin Wang, Walnut Creek, CA (US)

(72) Inventor: Tianxin Wang, Walnut Creek, CA (US)

(73) Assignee: Tianxin Wang, Walnut Creek, CA (US)

(21) Appl. No.: 16/004,415

(22) Filed: Jun. 10, 2018

Related U.S. Application Data
(63) Continuation-in-part of application No. 15/169,640, filed on May 31, 2016.
Application No. 15/169,640 is a continuation-in-part of application No. 15/373,483, filed on Dec. 9, 2016.
(60) Provisional application No. 62/517,994, filed on Jun. 12, 2017.

(51) Int. Cl. A61K 47/68; A61K 47/64; A61K 39/00; C07K 14/58

(52) U.S. Cl. CPC A61K 47/6897; A61K 47/6851; A61K 47/6425; A61P 35/00; C07K 14/58; A61K 47/6803; A61K 39/0011

(57) ABSTRACT

This disclosure provides reagent to treat tumor and cancer, and methods of using the same for treating tumor and cancer. One type of the reagent is pro-antibody, which is antibody that can be activated in tumor. Another type of the reagent is a conjugate of sialidase with affinity ligand that can bind to immuno cell surface or a conjugate of sialidase with affinity ligand that can bind to another antibody, therefore provide a sialidase based cancer immuno therapy strategy.

### CROSS-REFERENCE TO RELATED APPLICATIONS

This application claims priority to U.S. Provisional Patent Application 62/517,994 filed on Jun. 12 2017, and is a Continuation-In-Part application of U.S. patent application Ser. No. 15/169,640 filed on May 31, 2016, and a Continuation-In-Part application of U.S. patent application Ser. No. 15/373,483 filed on Dec. 9, 2016. The entire disclosure of the prior application is considered to be part of the disclosure of the instant application and is hereby incorporated by reference.

### BACKGROUND OF THE INVENTION

### Field of the Invention

The current invention relates to protein, peptide and enzyme modification for pharmaceutical applications and reagents to treat disease such as cancer; and methods of using the same for treating tumor and cancer.

### Background Information

Despite recent advances in tumor therapy of solid tumors such as antibodies, the need for more efficacious and cost-effective treatment options remains. Thermotherapy or more specifically hyperthermia is an appealing approach for the treatment of cancer, as, compared to chemotherapy or radiation therapy, fewer side effects are expected for a wide range of tumor diseases due to its physical mode of action. However, currently available modalities are still suboptimal and warrant improvement.

The U.S. Food and Drug Administration has approved several checkpoint inhibitors for the treatment of various cancers over the last decade. Checkpoint inhibitors work by exposing cancer cells that have hidden from the immune system. Cancer cells deceive immune cells by sending signals at certain checkpoints that indicate they are not harmful. If not for these checkpoints, T-cells would attack healthy cells. Immunotherapy drugs disrupt the cancer cells' signals, exposing them to the immune system for attack.

Researchers continue to search for new drugs, as well as combinations thereof with other known checkpoint inhibitor drugs, for use in treating tumors, as improved treatment results for patients with breast cancer, gastric cancer and other advanced cancers are needed.

### BRIEF DESCRIPTION OF THE DRAWINGS

FIG. 1 shows the structure and activating mechanism of self assembly probody

FIG. 2 shows examples of self assembly probody with Fc modifier

FIG. 3 shows an embodiment of self assembly probody with Fc modifier

FIG. 4 shows illustrates the mechanism and an embodiment of a pro-antibody

FIG. 5 shows other formats of pro-antibody using steric hindrance masking moiety.

FIG. 6 shows the activation mechanism of antibody drug conjugate based probody.

FIG. 7 shows formats of antibody drug conjugate based probody using steric hindrance masking moiety.

FIG. 8 illustrates the mechanism and an embodiment of bi/tri-specific antibody type probody

FIG. 9 shows an example of sialidase prozyme to treat cancer, which can be activated by uPA in the tumor to selectively cleave sialic acid on the tumor cells

FIG. 10 shows steric hindrance based masking of sialidase to generate a sialidase prozyme

FIG. 11 shows the scheme of sialidase conjugated with affinity ligand to bind with antibody drug to remove cancer surface sialic acid

FIG. 12 shows exemplary formats of the fusion protein contain sialidase and antibody/antibody fragment

FIG. 13 shows two or three full antibodies are linked with one or more linker such as a PEG or peptide linker to generate bi/tri-specific antibody to treat cancer

FIG. 14 illustrate the strategies to prepare the bispecific antibody using full antibody

FIG. 15 shows exemplary formats of the linear polymer based bispecific antibody

FIG. 16 shows exemplary formats of nano particle or liposome based bispecific antibody

### DESCRIPTION OF THE INVENTIONS AND THE PREFERRED EMBODIMENT

The current invention discloses novel methods and reagents to treat cancer. The method to treat cancer is to give the patient in need with therapeutically effective amount of one or more of these reagents alone or in combination with other anti-cancer reagents in suitable formulation such as injection form.

One type of the novel reagent in the current invention is pro-antibody, which is antibody that can be activated in tumor. Another type of the novel reagent in the current invention is a conjugate of sialidase with affinity ligand (e.g. antibody or aptamer) against immuno cell surface marker or against another antibody, which will provide a sialidase based cancer immuno therapy.

The current invention discloses novel strategy for antibody or aptamer construction, which can be activated by enzyme; they are called pro-antibody or probody and protamer respectively. Protamer is aptamer that can be activated by enzyme.

Probody (e.g. those developed by Cytomx Inc.) is pro-antibody that can be activated (having binding affinity to antigen after activation) by enzyme. US patent/patent applications U.S. Pat. No. 8,529,898, US 2010/0189651 (U.S. Ser. No. 12/686,344), US20130315906 (U.S. Ser. No. 13/872,052) and US20140010810 (U.S. Ser. No. 13/923,935) disclosed antibody construction called probody that can be activated by enzyme.

The pro-antibody (or called probody) in these prior art are activatable binding polypeptides (ABPs, e.g. antibody), which contain a target binding moiety (TBM), a masking moiety (MM), and a cleavable moiety (CM) are provided. Activatable antibody compositions, which contain a TBM containing an antigen binding domain (ABD), a MM and a CM are provided. Furthermore, ABPs which contain a first TBM, a second TBM and a CM are provided. The ABPs exhibit an “activatable” conformation such that at least one of the TBMs is less accessible to target when uncleaved than after cleavage of the CM in the presence of a cleaving agent (e.g. enzyme) capable of cleaving the CM. Further provided in the prior art are libraries of candidate ABPs, methods of screening to identify such ABPs, and methods of use. Further provided are ABPs having TBMs that bind VEGF, CTLA-4, or VCAM, ABPs having a first TBM that binds VEGF and a second TBM that binds FGF, as well as compositions and methods of use. The prior art disclosure provides modified antibodies which contain an antibody or antibody fragment (AB) modified with a masking moiety (MM). Such modified antibodies can be further coupled to a cleavable moiety (CM), resulting in activatable antibodies (AAs), wherein the CM is capable of being cleaved, reduced, photolysed, or otherwise modified. AAs can exhibit an activatable conformation such that the AB is more accessible to a target after, for example, removal of the MM by cleavage, reduction, or photolysis of the CM in the presence of an agent capable of cleaving, reducing, or photolysing the CM.

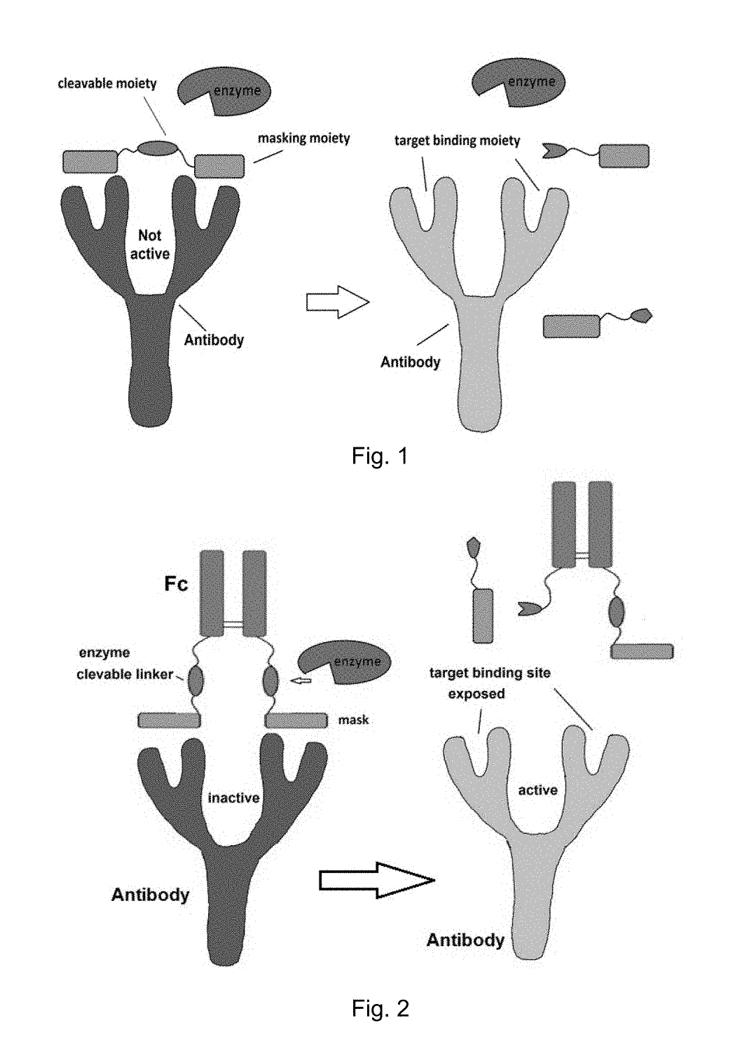

U.S. patent application Ser. Nos. 15/169,640 and 15/373,483 from the current inventor also disclose novel probody and protamer designs. These inventions disclose novel pro-antibody (probody) format. In the prior art from Cytomx, the masking moiety MM is covalently conjugated to the target binding moiety TBM (e.g. antibody, receptor, ligand for receptor such as VEGF). In Ser. Nos. 15/169,640 and 15/373,483 inventions, the difference is that the masking moiety MM is not covalently linked to the TBM (e.g. antibody, receptor, ligand for receptor such as VEGF). The cleavable moiety (CM) connect two MM instead of connecting the MM with the TBM in the prior art. Optionally a linker/spacer (e.g. a peptide or PEG) can be added between the MM and CM to allow optimal binding of two MM to the two Fab sites (or other binding moieties such as VEGF). The TMB such as antibody, MM and CM sequence can be essentially the same as these in the Cytomx prior arts disclosure except the linking between them is different as described above. The probody in the Ser. Nos. 15/169,640 and 15/373,483 invention is a bound complex instead of a single molecule as that in the Cytomx prior art. This strategy allows the use of the current available antibody or protein without the need to develop a new conjugate, therefore simplify the drug development process. The enzyme will cleave the CM and activate the TBM by exposing the previously blocked binding sites (example shown in FIG. 1).

Preferably antibody Fc or its fragment (e.g. single chain) can be connected to the MM (either by chemical conjugation or fusion/expression) to increase its half life (example shown in FIG. 2). Besides Fc tag, other half life extender (e.g. PEG, albumin, lipophilic tag, Xten, carboxyl-terminal peptide (CTP) of human chorionic gonadotropin (hCG)-beta-subunit) currently used to extend in vivo protein half life can also be attached to the MM covalently to reduce its in vivo inactivation/elimination. The antibody (or other TBM) can be conjugated with drugs as a targeted drug delivery system.

In one example (FIG. 3), Trastuzumab emtansine self-assembly probody is disclosed, which uses a MMP-9 substrate peptide LLGPYELWELSH (SEQ ID NO: 1) for tumor specific activation. LLGPYELWELSHGGSGGSGGSGGSVPLSLYSGGSGGSGGS (SEQ ID NO: 2) containing a HER2 mimic peptide, linker peptides and MMP-9 substrate peptide is fused with Fc, which forms a self assembly complex with Trastuzumab emtansine to block its binding affinity with HER-2 when no MMP-9 is present. The matrix metallopeptidase 9 (MMP-9) cleave the Fc-Mask peptide; release the active Trastuzumab emtansine (Kadcyla) to bind with HER2 on the tumor cell for targeted cancer therapy.

The current invention discloses novel strategy for pro-antibody construction used as reagent to treat cancer, which is antibody that can be activated by tumor enzyme. The similar strategy can also be used for other affinity ligand against tumor such as aptamer or antibody mimetics (such as affibody, affimer, examples of them can be found in en.wikipedia.org/wiki/Antibody_mimetic). The disclosed reagent can be used to treat tumor and cancer when being given to the patient in need. For example, the disclosed reagent can be administered to the patient (e.g. 10 mg˜500 mg intravenous injection daily or weekly or bi weekly or monthly) to treat tumor and cancer.

In the novel pro-antibody of the current invention, instead of the Fab (antigen binding domain) is masked (e.g. those from Cytomx format and prior arts), the effector portion/region of antibody is masked or blocked or deactivated. The effector of the antibody will be activated in tumor mostly with little or no activation in healthy tissue, therefore reduce off target of antibody drug. The advantage is that antibody will not be cytotoxic until it reaches the tumor, a potentially more effective targeting mechanism than the Cytomx format because activated antibody may escape the tumor quickly causing off target binding.

Suitable effectors include 1) the Fc region of antibody that induce ADCC (antibody-dependent cell-mediated cytotoxicity) and/or CDC (Complement-dependent cytotoxicity), which include NK cell/macro phage binding domain and complement binding domain 2) the toxin part in the antibody drug conjugate and 3) the cytotoxic cell binding Fab part and/or Fc part in the bi-specific antibody.

The construct of the novel pro-antibody in the current invention contains a target binding moiety (TBM), a masking moiety (MM), and a cleavable moiety (CM) similar to the prior probody constructs. The difference is that 1) the target binding moiety can be either an antibody used in probody or a probody itself (e.g. those described by Cytomx or in the prior applications by the current inventor) and 2) a masking moiety is used to mask or block or deactivate one or more said effector described above. The cleavable moiety sequence can be adopted from prior arts. The cleavable moiety connects the binding moiety (TBM) with the masking moiety (MM). In some embodiments, the cleavable moiety is connected with the antibody at the Fc portion of the antibody (e.g. at Fc's C terminal, heavy chain). In some embodiments, it is fused to the N terminal of the antibody of heavy chain or N terminal of light chain or C terminal of light chain.

The length and sequence of the cleavable moiety can be adjusted to allow the optimal masking or binding of masking moiety with the said effector to effectively deactivate it. In some embodiments, it is preferred that the masking moiety does not significantly affect the binding of antibody with FcRN recptor. Linkers such as a peptide or synthetic polymer such as PEG can be used to connect TBM, MM and CM. TBM, MM and CM can also be connected directly without linker.

The masking moiety can contain either a large size moiety (such as high MW PEG or Xten peptide from Amunix or PAS peptide from XL-protein GmbH or other high MW polymer) that provide steric hindrance to mask the effector region; or a affinity ligand that can bind to the effector region to block its activity or the combination of the above.

The masking moiety can be a masking peptide that can bind with Fc to block cytotoxic activity such as phage displayed peptide or those derived from natural ligand for Fc, e.g. Protein A or Fc receptor (for example CD16/FcγRIII) or their Fc binding domain. Examples of affinity ligand that can bind to the effector region include protein A, protein G, their binding domain to Fc, antibody or its fragment such as Fab against Fc, aptamer that bind to Fc as well as peptide that binds to Fc. There are many synthetic peptides that can bind with Fc available from literatures and publications, for example those disclosed in Novel peptide ligand with high binding capacity for antibody purification doi: 10.1016/j.chroma.2011.12.074; Screening of peptide ligands that bind to the Fc region of IgG using peptide array and its application to affinity purification of antibody, Biochemical Engineering Journal Volume 79, 15 Oct. 2013, Pages 33-40. These can be readily adopted by the skilled in the art for the current invention.

The cleavable moiety (CM) of the current invention can be readily adopted from the probody in the prior arts by the skilled in the art, for example it can contains a tumor produced protease/peptides cleavable substrate sequence. The whole construct of the current invention can be either expressed as a recombinant protein or chemically conjugated together. Examples of the tumor enzyme can be found in the probody from Cytomx Inc., such as legumain, plasmin, TMPRSS-3/4, MMP-9, MT1-MMP, cathepsin, caspase, human neutrophil elastase, beta-secretase, uPA, or PSA. The cleavable moiety used in the probody from Cytomx can be used as the cleavable peptide sequence. For example, metalloprotease 9 (MMP-9) substrate cleavage site VPLSLYS (SEQ ID NO: 3) added with flexible glycine-serine rich linker can be used as cleavable moiety for the construct as MMP-9 is frequently over expressed in epithelial malignancies.

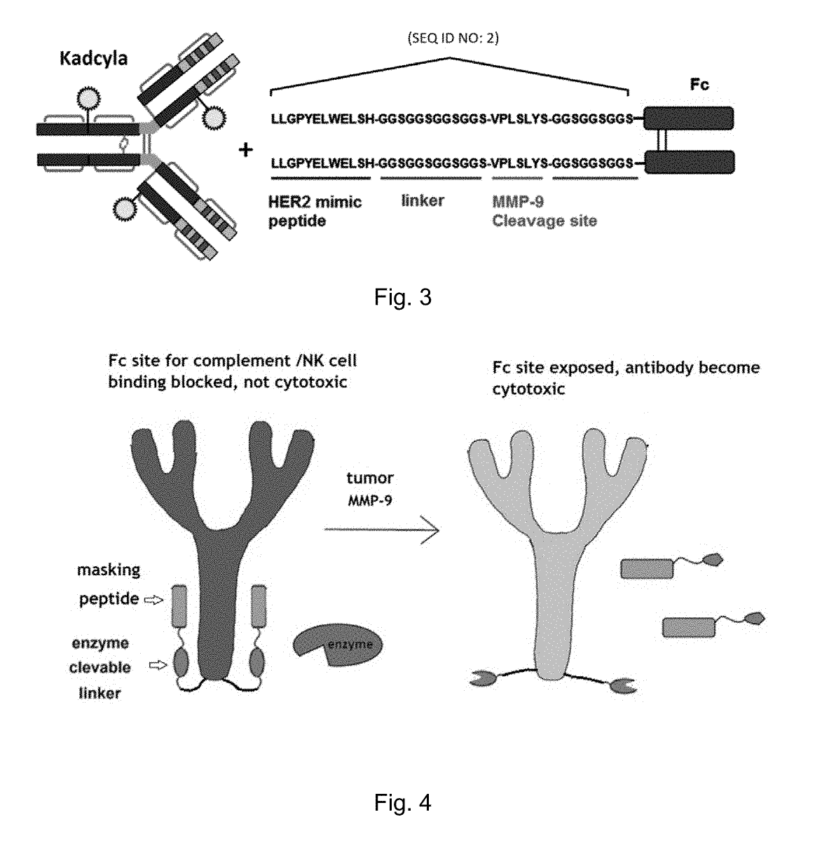

FIG. 4 illustrates the mechanism and an embodiment of a pro-antibody of the current invention. The construct in FIG. 4 is a recombinant protein in which the cleavable moiety is connected to the C terminal of the antibody's Fc and then connected to a masking peptide's N terminal covalently. After it enters tumor, the enzyme cleavable linker part (enzyme substrate) is cleaved by the MMP-9 and release the masking peptide from binding to the Fc of the antibody, convert the previous non-cytotoxic antibody to a cytotoxic antibody.

In one example of the above construct, the antibody (TBM) is Trastuzumab or it's probody. The masking moiety is protein A, the CM is GGSGGSGGSGGSGG-GSGGSVPLSLYS-GGSGGSGGSGGSGG- (SEQ ID NO: 4). CM contains a linker peptide sequence GGSGGSGGSGGSGG- (SEQ ID NO: 5) and a MMP-9 substrate peptide sequence. CM is fused with Fc and MM is fused with CM by recombinant technology. The linker sequence in CM can be repeated several times (e.g. 2-10 times before and after the MMP-9 substrate peptide sequence to allow optimal enzyme cleavage efficacy.

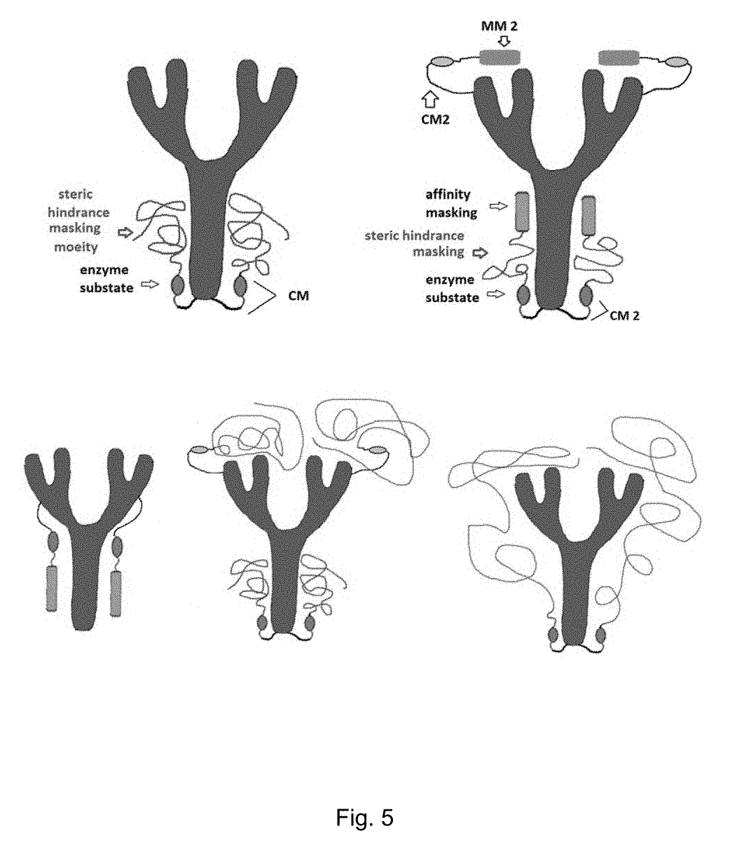

FIG. 5 shows other formats of the current invention. The masking moiety can be either affinity ligand that can bind to Fc or bulky high MW polymer that block the Fc region by steric hindrance instead of affinity binding to Fc, or their combination. The masking moiety containing a large size moiety can be either synthetic polymer such as PEG (e.g. 10 KD˜100 KD PEG) or recombinant peptide (e.g. a polypeptide with MW 20 KD˜200 KD) such as the Xten peptide used in ProTia platform from Amunix or PAS peptide from XL-protein GmbH. The examples in FIG. 5 below show the steric hindrance based masking and the combination of steric hindrance and affinity based masking (e.g. protein A). The antibody can be either active antibody or probody. The fusion can be either on the N terminal of the antibody or C terminal of the antibody from either heavy chain or light chain.

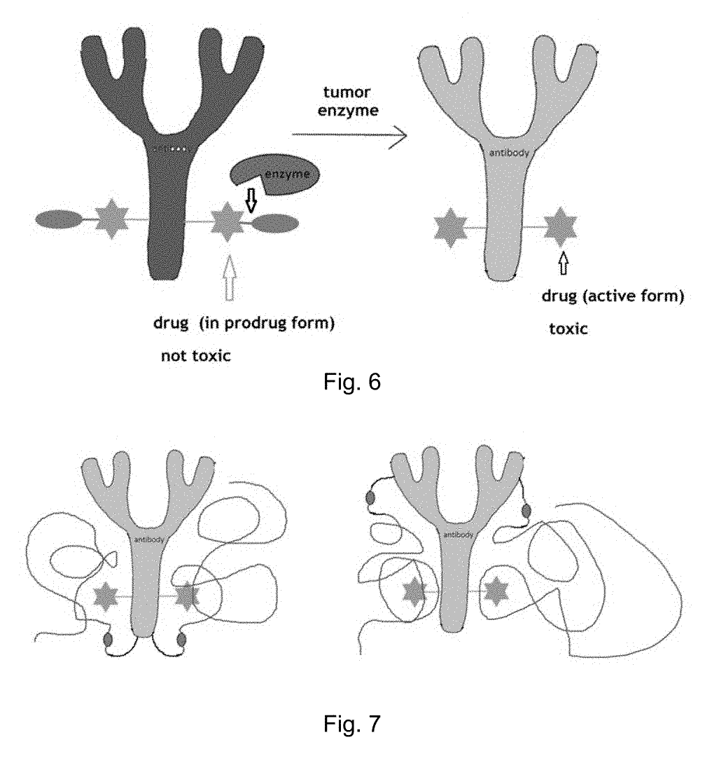

Similar strategy can also be used in antibody drug conjugate. The toxin is conjugated with a blocking/deactivating moiety that can mask the activity of the toxin via a CM moiety. The cleavage of CM by tumor enzyme restores the activity of the toxin as shown in FIG. 6.

Alternatively the toxin or the antibody is conjugated with a bulky MM via a CM, which blocks or reduces either the toxin activity or the endocytosis of the antibody. The cleavage of CM by tumor enzyme removed the bulky MM to restore the activity of the toxin or the endocytosis of the antibody. Examples are shown in the FIG. 7.

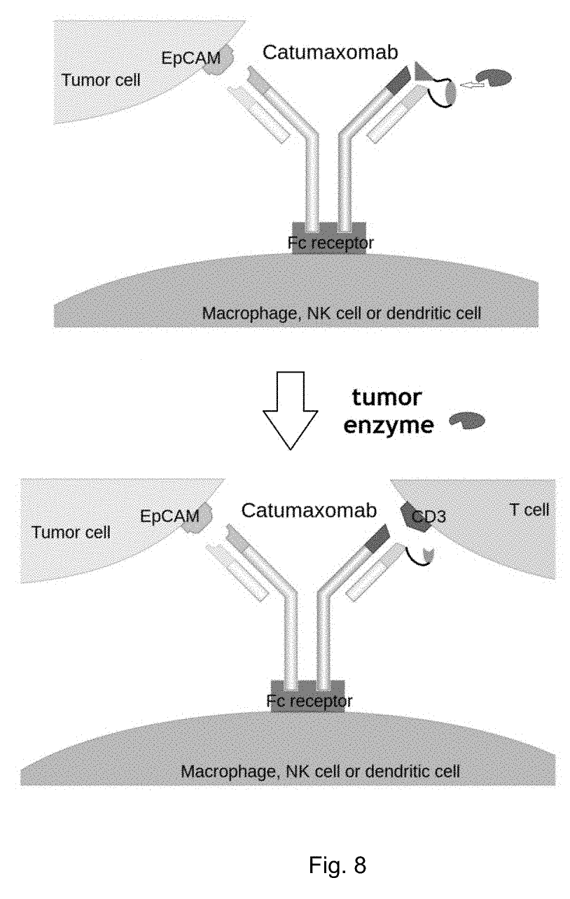

The probody strategy can also be applied in the bi-specific antibody or tri-specific antibody. The cytotoxic (e.g. T cell, NK cell) cell binding Fab part or affinity binder part can be masked with a MM conjugated to the bi/tri-specific antibody or bi/tri-specific affinity ligand via a CM as shown in the exemplary FIG. 8. Alternatively cancer cell binder part can be masked with a MINI conjugated to the bi/tri-specific antibody or bi/tri-specific affinity ligand via a CM or both the cancer cell binder part and cytotoxic part can be masked with a MM via a CM. Similarly, the current effector region blocking pro-antibody strategy can also be applied in the bi-specific antibody or tri-specific antibody, in which its Fc portion can be masked and then activated by tumor enzyme. The combination of probody and the current effector region blocking pro-antibody format can also be applied in bi-specific antibody or tri-specific antibody. Similar strategy can also be applied to other bi/tri specific affinity ligand such as aptamer based.

The construction of these probodies can be made readily by adopting the methods and protocols described in the cited prior arts and those described in the literatures by a skilled in the arts using well know recombinant technology and/or site specific conjugation technologies.

This strategy can also be used to provide therapeutic enzyme conjugate containing an enzyme and a affinity ligand such as antibody to provide targeted delivery to certain target such as tumor, therefore provides better target specificity. For example, the affinity ligand can bind with certain cell or pathogen surface marker and the enzyme can produce certain biological effect to the cell or pathogen. When there is no target cell/pathogen present, the enzyme conjugate is not localized, when the maker bearing cell/pathogen is present, the enzyme conjugate bind with the cell/pathogen and the local concentration enzyme is higher therefore become more active, produce stronger therapeutical effect to the cell or pathogen.

U.S. patent application Ser. Nos. 15/169,640 and 15/373,483 from the current inventor disclose novel strategy for enzyme construction which is called Cleavage Based Prozyme. Cleavage Based Prozyme is an activatable enzyme, which contains an active enzyme moiety (or a catalytic domain of an enzyme) conjugated with enzyme inhibitor moiety via a second enzyme (or other condition such as low pH or reducing environment) cleavable moiety (CM), a mechanism similar to probody. When there is no second enzyme or suitable cleavage condition, the active enzyme moiety binds with the enzyme inhibitor moiety or is blocked by the enzyme inhibitor moiety, therefore has low or no activity. When there is second enzyme or other cleavage condition, the enzyme cleavable moiety is cleaved to release the enzyme inhibitor from the enzyme, therefore the enzyme is activated to show high catalytic activity. The second enzyme can be either the same as the activatable enzyme or an enzyme with different catalytic activity.

The cleavable moiety is covalently coupled to the enzyme; the cleavable moiety is also coupled with an enzyme inhibitor (e.g. a molecule that can mask the enzyme catalytic center). In one example, glutathione S-transferase- PEG 20-CCCAAA-fluorescein-3′ is made by coupling 5′-PEG 20-CCCAAA-fluorescein-3′ having a —COOH group at the PEG end with the amine group on the enzyme using EDC. -CCCAAA is DNA fragment which can be cleaved by DNase. Fluorescein is an inhibitor of glutathione S-transferase. The resulting conjugate has low enzyme activity when there is no DNase and has high enzyme activity when DNase is present.

This strategy can be used to provide therapeutic enzyme conjugate that become activated enzyme when it is close to a target having the second enzyme, therefore provides better target specificity. For example, the second enzyme can be on the surface of or inside certain cell or pathogen and the enzyme can produce certain biological effect to the cell or pathogen. When there is no target cell/pathogen present, the enzyme is inactive, when the second enzyme bearing cell/pathogen is present, the enzyme conjugate will be cleaved by the cell/pathogen and the enzyme become active, produce therapeutical effect to the cell or pathogen. In one example, the cleavable moiety is a special peptide sequence that can be cleaved by a protease, the enzyme is an esterase that can convert an anti cancer prodrug to its active form. This prozyme can be used to selectively inactivate the said protease rich cancer cells.

Furthermore, the prozyme can be conjugated to or fused to an affinity ligand (e.g. an antibody) to provide further selectivity. In one example, the antibody is an antibody against HER2, therefore the Prozyme-antibody conjugate can be used to kill HER-2 positive cancer cells. In one example, the cleavable moiety and the linker connecting antibody with the enzyme (e.g. those currently used in ADC drugs) are substrate of the enzyme in lysosome. After endocytosis, the prozyme-antibody conjugate in the lysosome is cleaved to release the active enzyme to kill the cancer cell. Hydrophilic carbon chain can be introduced into the conjugate to help breaking the lysosome membrane.

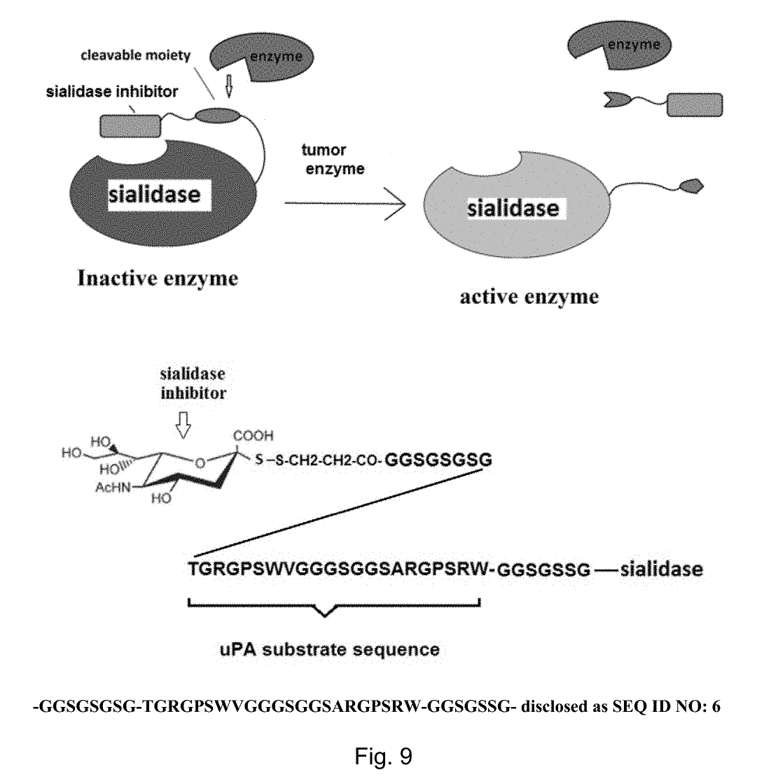

In some embodiments, the enzyme activatable prozyme strategy is applied to sialidase (neuraminidase). Tumor cell surface has high density of sialic acid, which protects the tumor cell from attack of the immune system and antibody drugs. Removing the cancer cell surface sialic acid can improve the efficacy of immune therapy and immune cell cytotoxicity against tumor cell. Antibody-sialidase conjugate can remove tumor cell surface sialic acid, improves the complement activation and ADCC of antibody drug (e.g. Herceptin) by activating NK cell. The prozyme strategy can be applied to sialidase for cancer therapy. The prozyme can be administered to the patient (e.g. 10 mg˜500 mg intravenous injection daily or weekly) to increase their immune response against cancer cells from immune cell as well as antibody drugs. As shown in FIG. 9, the sialidase is covalent linked with a flexible linker, the linker contains one or more tumor enzyme cleavable peptide sequence or non-peptide substrate (e.g. an oligosaccharide), the linker is further linked with a sialidase inhibitor. The whole structure can be either expressed as a recombinant protein or chemically conjugated together. Examples of the tumor enzyme can be found in the probody from Cytomx Inc., such as legumain, plasmin, TMPRSS-3/4, MMP-9, MT1-MMP, cathepsin, caspase, human neutrophil elastase, beta-secretase, uPA, or PSA. The cleavable moiety used in the probody from Cytomx Inc. can be used as the cleavable peptide sequence. The flexile linker contains flexible peptide sequence or other flexible polymer (e.g. PEG) at optimal length to allow the sialidase inhibitor bind with the sialidase when the linker is not cleaved. The sialidase can be either human sialidase or bacterial sialidase or virus sialidase such as flu sialidase, V. Cholerae sialidase, NEU1, NEU2, NEU3 and NEU4. FIG. 9 shows an example of sialidase prozyme to treat cancer. It can be activated by uPA in the tumor therefore selectively cleave the sialic acid on the tumor cells. It contains a sulfur substituted sialic acid as sialidase inhibitor, which connect to a flexible linker with disulfide bond. The flexible linker contains an uPA cleavable sequence. Another end of the linker is connected to the N terminal of sialidase either by chemical conjugation or expression. An example of the flexible linker for uPA is:

(SEQ ID NO: 6)

-GGSGSGSG-TGRGPSWVGGGSGGSARGPSRW-GGSGSSG-

The GS rich peptide region before and/or after the uPA substrate region in the above sequence can be repeated (e.g. 5˜20 times) to give the optimal linker length to allow the intra molecular binding of the inhibitor with the sialidase.

When the therapeutical antibody is an antibody against pathogens such as bacterial, the sialidase conjugate in the current invention can also be used to increase the efficacy of treating pathogens by removing the sialic acid on the pathogen surface.

Sialidase and its conjugate with other molecules such as affinity ligand (e.g. antibody including bi specific antibody) can be used to treat cancer, pathogen infection including bacterial and virus. Many pathogens have sialic acid on their surface to prevent host killing/clearing them. Sialic acid on the host cell surface is also a target for pathogen entry/infection (e.g. flu virus). Cleaving sialic acid from host cell or pathogen surface with sialidase or its derivative can be used to treat pathogen infection. Removing sialic acid from cancer cell and/or immune cell with sialidase or its derivative can also enhance the activity of immune cell against cancer cells. For example, removing the sialic acid on immune cell surface such as Dc cell with sialidase can improves antigen cross-presentation and boosts anti-tumor immune responses. Giving patient in need with therapeutically effective amount of sialidase or its derivative in suitable formulation such as injection form can be used to treat cancer and pathogen infection.

Prior U.S. patent application Ser. Nos. 15/169,640 and 15/373,483 from the current inventor disclose novel sialidase conjugate and the prozyme form of sialidase. The prozyme form of sialidase use sialidase inhibitor as masking moiety to block its activity before enzyme activation (e.g. tumor protease) in the prior arts. An alternative is to use bulky steric hindrance masking moiety to block its activity before enzyme activation, similar to those described in the above said pro-antibody. The construct can be the same as the above pro-antibody except the antibody is replaced with sialidase.

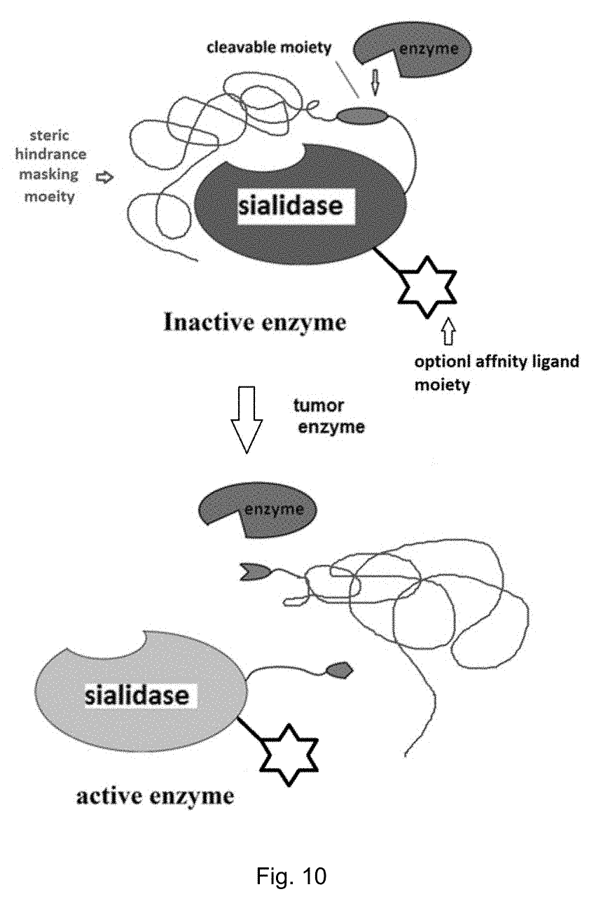

In some embodiments the masking moiety can be a bulky high MW polymer that block the sialidase catalytic region by steric hindrance instead of enzyme inhibitor, or their combination. The masking moiety containing a large size moiety can be either synthetic polymer such as PEG (e.g. 10 KD˜100 KD PEG) or recombinant peptide (e.g. a polypeptide with MW 20 KD˜200 KD) such as the Xten peptide used in ProTia platform from Amunix or PAS peptide from XL-protein GmbH. The masking moiety is connected to a cleavable moiety, which is then connected to the sialidase. The examples in FIG. 10 show an example of steric hindrance based masking of sialidase to generate a sialidase prozyme. The affinity ligand for cell surface molecule or pathogen surface molecule can also be conjugated to sialidase. The conjugate can be either a fusion protein or synthesized with chemical conjugation. The fusion can be either at the N terminal or C terminal of sialidase.

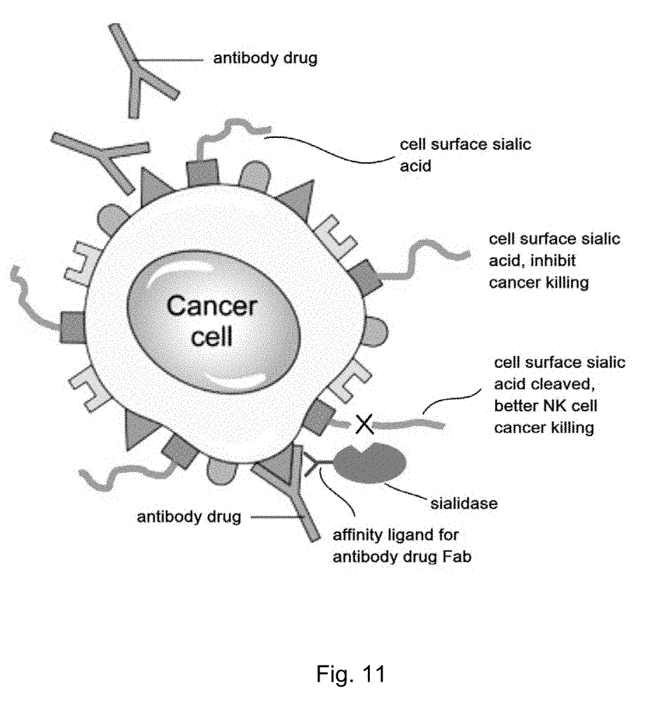

The sialidase (either active form or prozyme form) can also be conjugated with one or more affinity ligand to a therapeutical antibody (e.g. the therapeutical antibody is an antibody against cancer cell such as Herceptin). It will bind to the therapeutical antibody and cleave the sialic acid on the cancer cells once the anti cancer therapeutical antibody binds with cancer cells. This will provide targeted delivery of sialidase and increase therapeutical efficacy of the therapeutical antibody. It can be either pre-mixed with the therapeutical antibody to form the binding complex or injected to the patient separately to allow the sialidase-therapeutical antibody complex form in vivo. The affinity ligand can bind with either with Fc or Fab or F(ab′)₂ of the therapeutical antibody but should not block the binding of the therapeutical antibody to its target (non-neutralizing). Preferably the ligand binding with the therapeutical antibody should not inhibit the ADCC of antibody and should not inhibit complement activation. The therapeutical antibody binding ligand can either be peptide, antibody, antibody fragment, aptamer or small molecules. For example, when anti cancer therapeutical antibody is IgG containing humanized Fab, a non-neutralizing antibody or its F(ab′)₂ or Fab fragment against human IgG Fab region can be used to conjugate with sialic acid. In some embodiments, the antibody against human IgG Fab used for sialidase conjugation can be those used as secondary antibody against Fab in ELISA, for example, it can be Human IgG Fab Secondary Antibody (mouse anti human SA1-19255) from ThermoFisher or Mouse Anti-Human IgG Fab fragment antibody [4A11] (ab771) from Abcam or their F(ab)/Fab′/F(ab′)2 fragments. The anti-Human IgG Fab antibody or its fragment can be conjugated to the sialidase via a linker (e.g. PEG or flexible peptide) either chemically or by expression. The sialidase can be either active sialidase or the prozyme form sialidase. FIG. 11 shows the scheme of this kind of antibody binding sialidase conjugate.

In one example, the sialidase-antibody against Herceptin conjugate is prepared. Herceptin is a humanized IgG1 kappa antibody against HER2 receptor of cancer cells. Chicken IgY anti-human IgG1 Kappa light chain constant region (United States Biological catalogue # K0100-02) is used as antibody against Herceptin to prepare sialidase conjugate. In brief, Chicken IgY is first coupled to NHS-PEG4-azide (Thermo Fisher catalogue #26130) to introduce the azide linker to the IgY antibody on its lysine site using the coupling protocol provided by Thermo Fisher. Other site specific conjugation methods such as conjugating NH2-PEG6-azide to antibody with mTgase can also be used. The protocol can be found in the prior arts and readily adopted for the current synthesis. Separately, V. cholerae sialidase is nonspecifically functionalized on lysine residues with bicyclononyne-N-hydroxysuccinimide ester (BCN-NHS). After an overnight reaction, excess linker is removed with dialysis. Antibody adorned with the azide-functionalized linker is conjugated to BCN-functionalized V. cholerae sialidase via copper-free click chemistry. The desired conjugate is purified using a size-exclusion column. In another example, V. cholerae sialidase conjugated with avidin is mixed with biotinylated goat IgG anti human IgG Kappa light chain (IBL-America catalog #17249) at 1:1 molar ratio to form the sialidase-antibody against human IgG conjugate. The two sialidase-antibody human IgG conjugate in the above examples can be used to bind with anti cancer human IgG1 type therapeutical antibody drug to treat cancer.

Another type of the novel sialidase derivative (e.g. sialidase conjugate) of the current invention is a conjugate of sialidase with affinity ligand (e.g. antibody) that can bind to immune cell such as affinity ligand for immune cell surface molecule (e.g. its surface marker), which will provide a sialidase based cancer immunotherapy as well as a method and reagent to improve the activity of immune activity against pathogen infection. The sialidase in the conjugate can be either the active enzyme form or the prozyme form. The conjugate can be constructed as a fusion protein by recombinant technology or by chemical conjugation (preferably by site specific conjugation).

Immune cells for the current invention include leucocytes such as neutrophils, eosinophils, basophils, lymphocytes, and monocytes. Example of them include B cells, T cells, NK cells, macrophage, neutrophil and dendritic cell.

The immune cell surface marker can be cluster of differentiation type surface protein of immune cells. Examples of the immune cell surface marker can be either cell type specific such as KIR, CD3, sialic acid-binding immunoglobulin-type lectins or shared by many different types of immune cells (e.g. MEW II or FcR such as Cd16a). The binding of ligands used for the conjugate preferably have minimal negative impact on immune activity (e.g. cyto toxicity) of these cells against pathogens or cancer cells. When the ligand is affinity ligand for regulatory immune cell such as Treg, Breg and Tumor-associated macrophages, preferably the binding will not stimulate its immune inhibition activity, the affinity ligand can be a inhibitory or cytotoxic antibody against these regulatory immune cells.

The immune cell surface marker can be the stimulatory checkpoint receptor such as CD 27, CD28, CD 80, CD86, CD40, CD137, OX40, GITR, ICOS and etc. The immune cell surface marker can also be the inhibitory checkpoint receptor such as A2AR, BTLA, CTLA-4, KIR, LAG3, PD-1, TIM-3, VISTA and etc. In some embodiments preferably the affinity ligand to conjugate sialidase should not activate the inhibitory checkpoint receptor. In some embodiments, the affinity ligand can also be ligand targeting cancer specific marker such as VEGF, EGF, EGFR, VEGFR, cancer surface marker (e.g. CD47, sialic acid or poly sialic acid, GD2), cancer cell produced inhibitory ligand that can bind with inhibitory checkpoint receptor such as PD-L1, B7-H3, B7-H4 and etc.

Immuno cells are abundant in tumors. For example, Killer-cell immunoglobulin-like receptors (KIRs) is unique on NK cells and it inhibits NK cell activity similar to a check point inhibitor. Company such as Innate BMS is developing antibody against it to treat cancer (Lirilumab). Lirilumab or other antibody against KIRs can be conjugated with sialidase to treat cancer or pathogen infection. Other maker such as PD-1 and CTLA-4 on T cells can also be used. For example, FDA approved Ipilimumab (against CTLA-4) and Pembrolizumab (against PD-1) can be conjugated with sialidase. It is similar to bi-specific antibody format except one arm is antibody while another arm is enzyme instead of antibody. Anti PD-L1 antibody such as Atezolizumab can also be used to conjugate with sialidase, which will generate an conjugate that can bind to tumor cell and Treg. Besides these immune inhibitory cell surface markers, activating or non-regulatory marker such as CD3 on T cell or other markers on other immune cell can also be used. Non-antibody ligand such as peptide, aptamer or small molecule for these targets can also be used to conjugate with sialidase; there are several peptide and small molecule PD-1 or PD-L1 ligands available, which is disclosed in publications and literatures. For example, AUNP-12(Aur-012) peptide or those described in US Pat. Appl. 2011/0318373 or the PD-1 or PD-L1 ligand from Aurigene Discovery Technologies or the D-Peptide Antagonist (disclosed in DOI: 10.1002/anie.201506225) can be readily adopted to conjugate with sialidase to be used in the current invention. The term conjugate here covers both chemical conjugation and fusion by recombinant expression methods. The sialidase conjugate in the current invention has the general formula as: sialidase-(optional linker)-affinity ligand. Examples of linker can be found in prio arts and many publications such as PEG or a flexible peptide such as a short Xten like peptide. The sialidase can be mutated to introduce coupling site for site specific conjugation. There are many site specific conjugation methods that can be readily adopted form prior arts, publications and literatures.

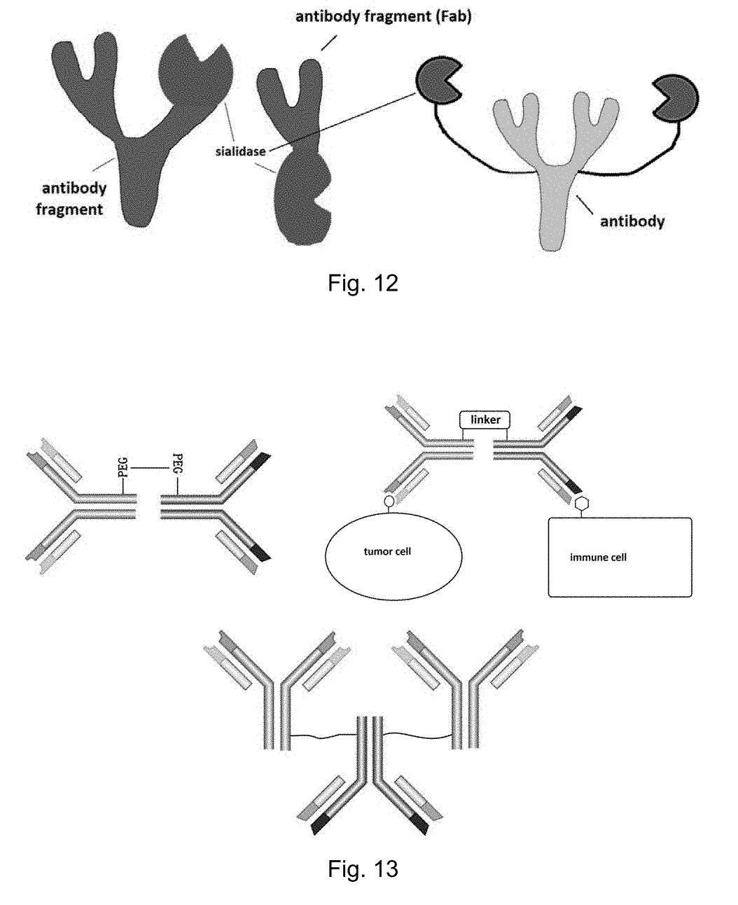

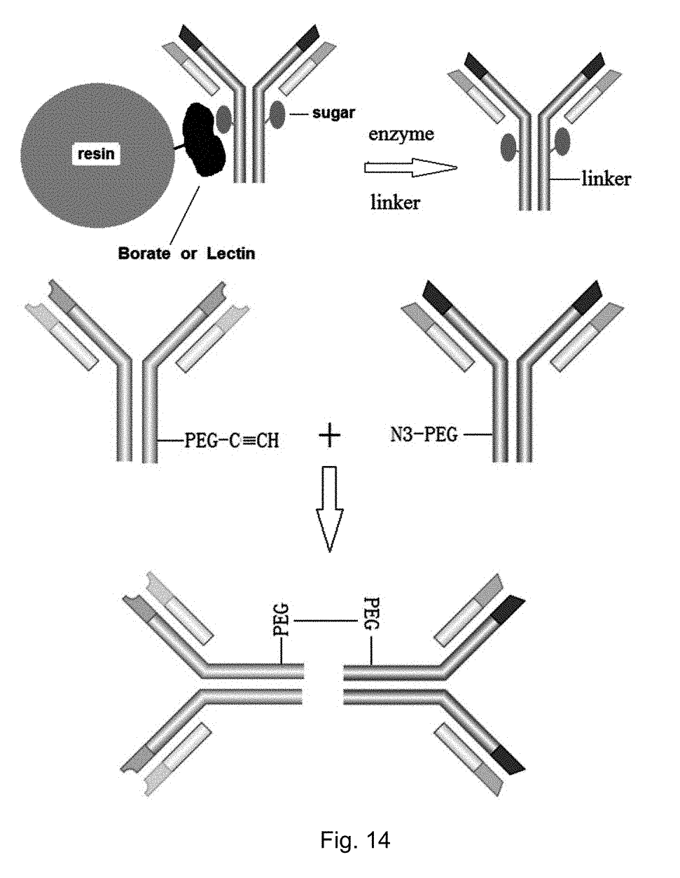

In one example, the sialidase-antibody against PD-1 (e.g. Pembrolizumab or the like) conjugate is prepared according to the protocol in Precision glycocalyx editing as a strategy for cancer immunotherapy, DOI: 10.1073/pnas.1608069113. The same conjugation protocol can be used except Pembrolizumab is used instead of Trastuzumab in this literature. In brief, Pembrolizumab bearing a C-terminal aldehyde tag is prepared. The functionalized antibody is first coupled to aminooxy-tetraethyleneglycol-azide (aminooxy-TEG-N3). Alternatively, NHS-PEG₅-azide can be used to introduce the azide linker to the antibody on its lysine site. Other site specific conjugation methods such as conjugating NH2-PEG₆-azide to antibody with mTgase can also be used. The protocol can be found in the prior arts and readily adopted for the current synthesis. Separately, V. cholerae sialidase is nonspecifically functionalized on lysine residues with bicyclononyne-N-hydroxysuccinimide ester (BCN-NHS). After an overnight reaction, excess linker is removed. Antibody adorned with the azide-functionalized linker is conjugated to BCN-functionalized V. cholerae sialidase via copper-free click chemistry. The desired conjugate is purified using a size-exclusion column. In another example, antibody against KIR (e.g. Lirilumab) is used instead of antibody against PD-1 to prepare the sialidase conjugate. In another example, antibody against Siglec-7 or siglec-9 is used instead. In another example, antibody or its fragment against sialic acid or hemagglutinin or lectin that can bind with sialic acid (e.g. Influenza hemagglutinin) is used instead. In another example, (D)NYSKPTDRQYHF-PEG₅-azide (a D-peptide that bind with PD-L1) is conjugated to BCN-functionalized sialidase with click chemistry to generate a (D)NYSKPTDRQYHF-linker-sialidase conjugate. The antibody can also be replaced with its fragment such as Fab or F(ab′)2. Fusion protein can also be constructed instead of chemical conjugation. FIG. 12 shows exemplary formats of the fusion protein contain sialidase and antibody/antibody fragment. The sialidase can be in prozyme form and the antibody can also be in probody or pro-antibody form or anti cancer bi specific antibody form.

These sialidase conjugates can be used as a reagent to treat cancer when being given to the patient in need. The conjugate can be administered to the patient (e.g. 10 mg-500 mg intravenous injection daily or weekly) to increase their immune response against cancer cells from immune cell. STAT inhibitor such as cucurbitacin and/or STAT Tyrosine kinase inhibitors (such as axitinib, sorafenib and sunitinib) can be administered to the patient at therapeutically effective amount to the patient with sialidase conjugate together to treat cancer and tumor. They can be formulated together or being give to the patient separately to boost their anti cancer efficacy.

The current invention also discloses novel Bi specific antibody and its application. They can be used to treat cancer, pathogens, immune disorders and as targeting delivery of vector (e.g. in retrovirus based gene therapy).

Bi specific antibody can be in traditional monomer format: multivalent homo Fab format with a suitable length flexible linker for higher affinity (not bi specific), hetero Fab format targeting two epitope sites of the different protein on the cell/microorganism to achieve higher affinity and hetero Fab format targeting two epitope sites of the target protein to achieve higher affinity.

Bi specific antibody can also be in dimer format or trimer or higher degree oligomer format: multivalent homo Fab format with suitable length flexible linker for higher affinity (not bi specific), hetero Fab format targeting two epitope sites of the target protein for higher affinity and hetero Fab format targeting two epitope sites of the different protein on the cell/microorganism for higher affinity. Construction of this type of Bi specific antibody can be achieved using boric affinity column or lectin affinity column for mono conjugation (boric affinity column or lectin affinity column can also be used for antibody purification).

It can also be hetero Fab format targeting two antigens of the different protein on the cell/microorganism for higher affinity. Similarly, the above approach can also be applied to bispecific antibody binding to two different antigens on the cell/pathogen. The bispecific antibodies with flexible proper length linkers can be made easily to get the optimal binding of two antigens simultaneously while traditional method is time consuming. Another format is to use bi specific antibody to target the two different epitopes on the same antigen, which will also significantly increase the binding affinity.

Similarly, bi specific antibody by linking two or more full size antibodies can also be used in above applications and formats and synthesized readily, which may offer higher stability and higher binding affinity as shown by IgA and IgM.

Construction of this type of Bi specific antibody can be achieved using borate affinity column or lectin affinity column for mono conjugation. This strategy is also useful for antibody purification. This design uses immobilized antibody to archive high yield mono labeling of the antibody, to eliminate the potential bi-labeled antibody (generating polymerized antibody).

Immobilized protein was used to make mono PEGlated protein previously. Ion exchange resin was used to immobilize the protein. However ion exchange resin may not work for antibody to block half of FC and the binding affinity is low, which may cause exchange between two sides.

This design uses affinity group targeting the carbohydrate on the antibody to selectively protect one FC conjugation site on the antibody to achieve the mono conjugation. Suitable affinity resins include borate based affinity solid phase support or lectin based affinity phase support (FIG. 7). When one side of the antibody is protected, the other side can be selectively modified (e.g. site specific conjugation using enzyme such as mTGase).

Borate is a carbohydrate chelators and borate based column is widely used in separating carbohydrate, many are commercially available (e.g. from Sigma). Different borate also has different affinity to different sugar. Lectins are carbohydrate-binding proteins, most are from plant, which is used as antivirus/bacterial drug for animals. Different lectin has selectivity for different carbohydrate. Lectin column is also used in studying carbohydrate. Lectin or borate based resin can also be a useful tool for large scale purification of antibody drugs during ADC labeling. They can also be used for protein mono labeling other than antibody if the protein has carbohydrate modification.

Using ADC made of BsAb against two makers on the target cell will increase the specificity of drug delivery.

Prior U.S. patent application Ser. Nos. 15/169,640 and 15/373,483 disclose novel bi-specific antibody and the make by linking two or more full size antibodies. The resulting bi-specific antibody can be used to increase binding affinity and specificity by binding two epitopes in one protein or one cell. It can also be used to treat cancer or other diseases by linking two or more antibodies binding to different targets (e.g. one binds with cancer cell and another binds with immune cell), which is the standard binding format employed by current bi-specific antibodies as shown in FIG. 13. Two or three full antibodies are linked with one or more linker such as a PEG or peptide linker. Suitable polymer that can be used as linker for protein conjugation are well know to the skilled in the art and can be readily adopted from publications and literatures.

The linker can be a synthetic polymer such as PEG or a flexible hydrophilic peptide (e.g. a peptide rich in Ser and Gly and Asp, 10˜50 AA). The length of the flexible linker can be optimized to allow the Fab of the resulting antibody bind to two different epitopes on the same target at the same time for bispecific Ab. Reactive amino acids (e.g. Cys or Gln) can be readily expressed in the liker for site specific conjugation of ADC, the flexibility of reactive linker allow optimal conjugation efficacy, the reactive flexible linker can be readily incorporated into many other formats of bispecific antibody. Besides the format described above, this reactive flexible linker strategy can be readily incorporated into many other formats of bispecific antibody.

The synthesis of this kind of bi-specific antibody can be achieved with conjugating the first antibody (e.g. antibody against CD3) with linker A and conjugating the second antibody (e.g. antibody against CD20 or EpCam) with linker B; and then connect linker A with linker B with the reactive groups on them (e.g. azide end of linker A and bicyclononyne end of linker B employing click chemistry). The key is to have at least one antibody conjugated with only one linker with each antibody. A full antibody is a symmetric structure with two light chains and two heavy chains, therefore normally generate the conjugate containing 2 or more linkers on each antibody. One strategy to generate an antibody carrying only one linker (mono conjugation/mono labeling) is to block/protect one of the two conjugating sites in the antibody and expose another only. This can be archived by conjugate the linker with immobilized antibody. During the conjugation the antibody is first immobilized on solid phase support (e.g. particles, columns), expose only one of the antibody's conjugation sites for coupling. First the antibody is immobilized on an affinity support and then the conjugation is performed. The affinity support can be poly styrene particles, ion exchange particles as well as solid phase support (e.g. resins or particles) coated with affinity ligand for antibody. Suitable affinity ligand includes borate, lectin, protein A, protein G and other affinity ligand that can bind with Fc or Fab of the antibody. Preferably the affinity ligand conjugated to solid phase support can mask one of the coupling sites of the antibody. For example, mono conjugation can be achieved using borate affinity resin/column or lectin affinity resin/column. This strategy is also useful for antibody purification. This design uses immobilized antibody to archive high yield mono labeling of the antibody, to eliminate the potential bi-labeled antibody (generating polymerized antibody). Hydrophobic binding resin or ion exchange resin can also be used to immobilize the antibody. Protein A or protein G coated resin/columns can also be used to immobilize antibody for mono conjugation. One strategy is to use affinity group targeting the carbohydrate on the antibody to selectively protect one of the two FC conjugation sites on the antibody to achieve the mono conjugation. Suitable affinity resins include borate based affinity solid phase support or lectin based affinity solid phase support. When one side of the antibody is protected, the other side can be selectively modified (e.g. site specific conjugation using enzyme such as mTGase). Borate is a carbohydrate chelators and borate based column is widely used in separating carbohydrate, many are commercially available (e.g. from Sigma). Different borate also has different affinity to different sugar. Lectins are carbohydrate-binding proteins, most are from plant, which is used as antivirus/bacterial drug for animals. Different lectin has selectivity for different carbohydrate. Lectin column is also used in studying carbohydrate. Lectin or borate based resin can also be a useful tool for large scale purification of antibody drugs during ADC labeling. They can also be used for protein mono labeling other than antibody if the protein has carbohydrate modification. Another strategy is to mix the antibody with soluble affinity ligand (e.g. lectin, protein A, protein G) at 1:1 to 1:1.5 molar binding ratio, therefore most antibody either have one conjugation site exposed or no conjugation site exposed, then the conjugation with linker is performed to generate mostly mono labeled antibody.

A third strategy is to use low molar ratio of linker (e.g. 1:1 between antibody and linker) to achieve mostly mono labeled antibody. This method is mostly suitable for thio bridge type site specific conjugation such as ThioBridge™, c-Lock™/K-Lock™ and Igenica's dithiopyridylmaleimide based conjugation method. One can adjust the -s-s- bond cleavage condition to allow only one -s-s- in the antibody becomes broken for conjugation or use low molar ratio of thio bridge forming reagent to generate only one labeling in each antibody. In one example, antibody is first immobilized on Phenyl Borate based Cellufine PB Affinity resin or protein A affinity resin or protein G affinity resin, next NH2-PEG₃-azide or NH2-Gly-Gly-azide is added to the immobilized antibody to perform the conjugation with mTGase. The reaction condition and protocol can be adopted from those described in US patent application U.S. Ser. No. 15/317,907. Other site specific conjugation method can also be employed, such as PNGase/mTGase conjugation method from innate pharma, GlycoConnect™ conjugation method from SynAffix, formylglycine-generating enzyme based conjugation, 6-Thiofucose based conjugation, extra cysteine or unnatural amino acids based site-directed conjugation via these gene engineering method and etc. After the conjugation, un-reacted reagents are removed and the mono labeled antibody is eluted from affinity resin for the next conjugation with the second mono labeled antibody (to generate a di-antibody type bi-specific antibody) or di-labeled antibody (to generate a tri-antibody bi-specific antibody).

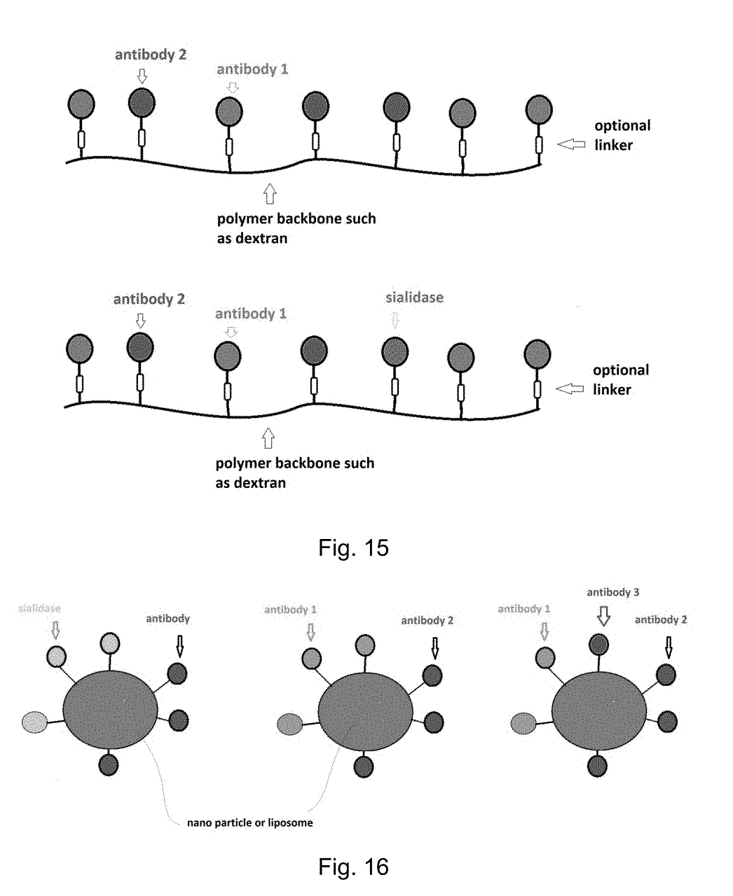

Multiple different antibodies or their fragments (e.g. Fab, F(ab′)₂) can also be conjugated/immobilized/coated to a carrier to generate a bi-specific antibody like structure to have similar activity of bi-specific antibody. The carrier can be a linear polymer such as dextran. In one example 2-8 two types of antibody is conjugated to a 10 KD dextran. It can also be formed by affinity binding, for example, dextran is first conjugated with avidin and mixture of biotinylated antibodies is added to form the polymer backbone based bi-specific antibody mimetic. FIG. 15 shows exemplary formats of the linear polymer based novel bispecific antibody. Sialidase can also be incorporated to the polymer carrier based bi-specific antibody.

The carrier can also be nano particle or liposome as illustrated in FIG. 16. Similarly multiple two or more different antibodies or their fragments can be conjugated to one particle surface or one liposome surface, providing the surface are labeled with reactive groups. Different antibodies or their fragments can be conjugated to the surface using the same chemistry or different chemistry to provide precise control of conjugation. The conjugation can also be performed using the mixture of different antibodies or conjugating different antibody sequentially. Alternatively, the surface can be coated with affinity ligand for antibody (e.g. protein A for unmodified antibody or avidin for biotinylated antibodies) and the immobilization of antibody or their fragments is performed by affinity binding. The antibody or their fragments can also be conjugated with lipid like molecule and then being inserted to lipid layer to form the desired antibody coated liposome. There are many reactive nano particle (e.g. NHS ester coated, maleimide coated) or affinity ligand (e.g. protein A coated) coated nano particle commercially available for the current invention. In some embodiments, the size of the nano particles or liposomes is between 5 nm˜1000 nm; the antibody or their fragments on each nano particle or liposome is between 4˜10000 copies.

Similarly, sialidase or its derivative can also be co immobilized with affinity ligand (e.g. one or more type of antibody or their fragments against cancer cell or immune cell) on the surface of nano particle or liposome.

Molecules (reagents) described herein can be administered as a pharmaceutical or medicament formulated with a pharmaceutically acceptable carrier. Accordingly, the Molecules may be used in the manufacture of a medicament or pharmaceutical composition. Pharmaceutical compositions of the invention may be formulated as solutions or lyophilized powders for parenteral administration. Powders may be reconstituted by addition of a suitable diluent or other pharmaceutically acceptable carrier prior to use. Liquid formulations may be buffered, isotonic, aqueous solutions. Powders also may be sprayed in dry form. Examples of suitable diluents are normal isotonic saline solution, standard 5% dextrose in water, or buffered sodium or ammonium acetate solution. Such formulations are especially suitable for parenteral administration, but may also be used for oral administration or contained in a metered dose inhaler or nebulizer for insufflation. Molecules may be formulated to include other medically useful drugs or biological agents. The molecules also may be administered in conjunction with the administration of other drugs or biological agents useful for the disease or condition to which the invention compounds are directed. Besides IV or SQ or IM injection, direct injection to target site such as intratumoral injection can also be employed.

In certain embodiments, the present disclosure is directed to a method of treating and/or inhibiting a tumor and its metastasis, comprising administering to a patient in need thereof a therapeutically effective amount of a conjugate or a pharmaceutical composition as described herein.

The disclosure relates to methods of treating cancer. Accordingly, provided herein is a method of treating and/or inhibiting a solid tumor, comprising administering to a patient in need thereof a therapeutically effective amount of the said reagent, a formulation or pharmaceutical composition as described herein. The reagent, a formulation or pharmaceutical composition as described herein can be injected intratumorally to treat the cancer. In certain embodiments, the treating and/or inhibiting comprises preventing metastasis of the tumor. In other embodiments, the method comprises administering a therapeutically effective amount of an immune check point inhibitor, such as T lymphocyte antigen 4 (CTLA4) blocking antibody, PD-1 blocking antibody, PD-L1 blocking antibody, ipilimumab, tremelimumab, atezolizumab, nivolumab or pembrolizumab, or a combination thereof.

As employed herein, the phrase “an effective amount,” refers to a dose sufficient to provide concentrations high enough to impart a beneficial effect on the recipient thereof. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet, and general health of the subject, and like factors well known in the medical arts and sciences. Various general considerations taken into account in determining the “therapeutically effective amount” are known to those of skill in the art and are described. Dosage levels typically fall in the range of about 0.001 up to 100 mg/kg/day; with levels in the range of about 0.05 up to 10 mg/kg/day are generally applicable. A compound can be administered parenterally, such as intravascularly, intravenously, intraarterially, intramuscularly, subcutaneously, or the like. Administration can also be orally, nasally, rectally, transdermally or inhalationally via an aerosol. The compound may be administered as a bolus, or slowly infused. A therapeutically effective dose can be estimated initially from cell culture assays by determining an IC50. A dose can then be formulated in animal models to achieve a circulating plasma concentration range that includes the IC50 as determined in cell culture. Such information can be used to more accurately determine useful initial doses in humans. Levels of drug in plasma may be measured, for example, by HPLC. The exact formulation, route of administration and dosage can be chosen by the individual physician in view of the patient's condition.

As used herein, the term “treating” refers to preventing, curing, reversing, attenuating, alleviating, minimizing, inhibiting, suppressing and/or halting a disease or disorder, including one or more clinical symptoms thereof.

As used herein, the term “composition” refers to a preparation suitable for administration to an intended patient for therapeutic purposes that contains at least one pharmaceutically active ingredient, including any solid form thereof. In certain embodiments, the composition is formulated as an injectable formulation. In certain embodiments, the composition is formulated as a film, gel, patch, or liquid solution. As used herein, the term topically refers to administering a composition non-systemically to the surface of a tissue (e.g., a tumor) and/or organ (internal or, in some cases, external; through a catheter) to be treated, for local effect.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the desired tissue or a tissue adjacent to the desired tissue.

As used herein, the term “formulated” or “formulation” refers to the process in which different chemical substances, including one or more pharmaceutically active ingredients, are combined to produce a dosage form. In certain embodiments, two or more pharmaceutically active ingredients can be coformulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “solution” refers to solutions, suspensions, emulsions, drops, ointments, liquid wash, sprays, liposomes which are well known in the art. In some embodiments, the liquid solution contains an aqueous pH buffering agent which resists changes in pH when small quantities of acid or base are added. In certain embodiments, the liquid solution contains a lubricity enhancing agent.

In the current application the “I” mark means either “and” or “or”. Unless otherwise defined, all technical and scientific terms used herein have the same meaning as commonly understood by one of ordinary skill in the art to which this invention belongs. All patents and publications mentioned in this specification are indicative of the level of those skilled in the art to which the invention pertains. All patents and publications are herein incorporated by reference to the same extent as if each individual publication was specifically and individually indicated to be incorporated by reference. The inventions described above involve many well-known chemistry, instruments, methods and skills. A skilled person can easily find the knowledge from text books such as the chemistry textbooks, scientific journal papers and other well-known reference sources.

1. A conjugate to treat cancer comprising a sialidase and an affinity ligand that can bind to immune cell surface.

2. The conjugate according to claim 1, wherein the immune cell is T cell.

3. The conjugate according to claim 1, wherein the immune cell is NK cell.

4. The conjugate according to claim 1, wherein the affinity ligand is antibody.

5. The conjugate according to claim 1, wherein the affinity ligand is aptamer.

6. A conjugate to treat cancer comprising a sialidase and an affinity ligand that can bind to a cancer cell binding antibody.

7. The conjugate according to claim 6, wherein the affinity ligand is antibody.

8. The conjugate according to claim 6, wherein the affinity ligand is aptamer.

9. A method of treating and/or inhibiting a tumor, comprising administering to a patient in need thereof a therapeutically effective amount of a conjugate comprising a sialidase and an affinity ligand that can bind to immune cell surface.

10. The method according to claim 9, wherein the immune cell is T cell.

11. The method according to claim 9, wherein the immune cell is NK cell.


SEQUENCE LISTING (6 sequences)

SEQ ID NO: 1 - protein, 12 aa, Artificial Sequence
  synthetic affinity peptide
LLGPYELWELSH

SEQ ID NO: 2 - protein, 40 aa, Artificial Sequence
  synthetic affinity peptide 2
LLGPYELWELSHGGSGGSGGSGGSVPLSLYSGGSGGSGGS

SEQ ID NO: 3 - protein, 7 aa, Artificial Sequence
  synthetic peptide linker
VPLSLYS

SEQ ID NO: 4 - protein, 40 aa, Artificial Sequence
  synthetic peptide linker 2
GGSGGSGGSGGSGGGSGGSVPLSLYSGGSGGSGGSGGSGG

SEQ ID NO: 5 - protein, 14 aa, Artificial Sequence
  synthetic peptide linker
GGSGGSGGSGGSGG

SEQ ID NO: 6 - protein, 37 aa, Artificial Sequence
  synthetic peptide linker
GGSGSGSGTGRGPSWVGGGSGGSARGPSRWGGSGSSG